(19) United States
(12) Patent Application Publication
(10) Pub. No.: US 2022/0051039 A1
(43) Pub. Date: Feb. 17, 2022

(54) BIOMETRIC IDENTIFICATION USING ELECTROENCEPHALOGRAM (EEG) SIGNALS

(71) Applicants: Mohammad Samin Nur Chowdhury, Roslyn, NY (US); Arindam Dutta, Tempe, AZ (US); Daniel W. Bliss, Scottsdale, AZ (US); Gene Brewer, Scottsdale, AZ (US); Christopher Blais, Phoenix, AZ (US); Matthew Robison, Dallas, TX (US)

(72) Inventors: Mohammad Samin Nur Chowdhury, Roslyn, NY (US); Arindam Dutta, Tempe, AZ (US); Daniel W. Bliss, Scottsdale, AZ (US); Gene Brewer, Scottsdale, AZ (US); Christopher Blais, Phoenix, AZ (US); Matthew Robison, Dallas, TX (US)

(73) Assignee: Arizona Board of Regents on behalf of Arizona State University, Scottsdale, AZ (US)

(21) Appl. No.: 17/402,049

(22) Filed: Aug. 13, 2021

Related U.S. Application Data
(60) Provisional application No. 63/065,117, filed on Aug. 13, 2020.

(51) Int. Cl. G06K 9/00; G06K 9/62; G06N 3/04; A61B 5/372; A61B 5/378; A61B 5/38

(52) U.S. Cl. CPC G06K 9/00885; G06K 9/00536; G06K 9/00523; G06K 9/624; G06K 2009/00939; G06N 3/04; A61B 5/372; A61B 5/378; A61B 5/38; G06K 9/0051

(57) ABSTRACT

Biometric identification using electroencephalogram (EEG) signals is provided. Embodiments are targeted for biometric applications, where an individual can be identified with a precision of over 99%, using sensed brain signals. In particular, a method is described which can extract unique biomarkers from EEG response signals to classify individuals, also referred to as simple visual reaction task-based EEG biometry (SVRTEB). A subject experiences a simple stimulus or task, and a multi-channel EEG response is recorded. Unique biomarkers are extracted from the recorded EEG response (e.g., as periodogram data points corresponding to different frequencies observed in the brain waves, which can be used to identify a person). A novel signal processing approach uses neural network-based architecture to analyze the EEG response and identify the subject. This signal processing architecture can be readily implemented on hardware and provides high accuracy, precision, and recall.

### RELATED APPLICATIONS

This application claims the benefit of provisional patent application Ser. No. 63/065,117, filed Aug. 13, 2020, the disclosure of which is hereby incorporated herein by reference in its entirety.

### FIELD OF THE DISCLOSURE

The present disclosure is related to biometric identification, and particularly to biometric identification based on brainwaves.

### BACKGROUND

Biometric authentication is used in a growing number of security systems, including physical and computing environments. Biometric authentication aims to identify an individual based on unique biological characteristics. Examples of existing biometrics used for authentication include fingerprinting, facial recognition, and retinal imaging.

Many biometrics are unsuited for people with specific diseases. In addition, many existing biometrics can be easily forged or collected without a person's consent. Relatedly, many existing biometrics are easy to interpret and can be used to determine a person's identity, raising significant privacy concerns.

### SUMMARY

Biometric identification using electroencephalogram (EEG) signals is provided. Embodiments are targeted for biometric applications, where an individual can be identified with a precision of over 99%, using sensed brain signals. In particular, a method is described which can extract unique biomarkers from EEG response signals to classify individuals, also referred to as simple visual reaction task-based EEG biometry (SVRTEB). A subject experiences a simple stimulus or task, and a multi-channel EEG response is recorded. Unique biomarkers are extracted from the recorded EEG response (e.g., as periodogram data points corresponding to different frequencies observed in the brain waves, which can be used to identify a person). A novel signal processing approach uses neural network-based architecture to analyze the EEG response and identify the subject. This signal processing architecture can be readily implemented on hardware and provides high accuracy, precision, and recall.

SVRTEB is universally applicable, as EEG signals can be acquired from all human brains. EEG biometrics cannot be easily forged or collected without consent. Because brainwaves carry unique biomarkers for an individual's response to external stimulus, the proposed model can identify individuals with an accuracy greater the 99%. In addition, SVRTEB can be implemented with commercially available devices, such that the entire process chain can be implemented on a field-programmable gate array (FPGA) or other readily available hardware for real-time identification. Finally, using EEG provides enhanced privacy as EEG signals are difficult to decode and decipher a subject's true identity.

An exemplary embodiment provides a method for identifying a human subject. The method includes obtaining EEG data for a human subject which is responsive to a stimulus; extracting a plurality of feature points from the EEG data; and analyzing the plurality of feature points to identify the human subject.

Another exemplary embodiment provides a biometric classification device. The biometric classification device includes an EEG sensor; a memory configured to store EEG data from the EEG sensor; and a processor. The processor is configured to: receive the EEG data for a human subject which is responsive to a stimulus; extract a plurality of feature points from the EEG data; and identify the human subject based on the plurality of feature points.

Another exemplary embodiment provides a biometric classification system. The biometric classification system includes a memory configured to store EEG data from an EEG sensor; and a processor. The processor is configured to: receive the EEG data for a human subject which is responsive to a stimulus; extract a plurality of feature points from the EEG data; and implement a neural network to identify the human subject from the plurality of feature points.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a schematic diagram of an exemplary visual response task used to extract unique biomarkers from electroencephalogram (EEG) signals according to embodiments described herein.

FIG. 2 is a diagram of exemplary electrode locations for recording multi-channel EEG data during the task of FIG. 1.

FIG. 3 is a flow diagram of a method for classifying an individual from EEG data according to embodiments described herein.

FIG. 4 is a graphical representation of exemplary multi-channel EEG data from the task of FIG. 1.

FIG. 5 is a graphical representation of a periodogram from five channels of the multi-channel EEG data of FIG. 3 representing five different brain lobes.

FIG. 6 is a schematic block diagram of an exemplary neural network architecture for classifying an individual from EEG data according to embodiments described herein.

FIG. 7 is a graphical representation of classification accuracy of one hundred randomized Monte Carlo simulations using embodiments described herein.

FIG. 8 is a flow diagram illustrating a process for identifying a human subject.

FIG. 9 is a block diagram of a biometric classification device suitable for implementing simple visual reaction task-based EEG biometry (SVRTEB) according to embodiments disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Biometric identification using electroencephalogram (EEG) signals is provided. Embodiments are targeted for biometric applications, where an individual can be identified with a precision of over 99%, using sensed brain signals. In particular, a method is described which can extract unique biomarkers from EEG response signals to classify individuals, also referred to as simple visual reaction task-based EEG biometry (SVRTEB). A subject experiences a simple stimulus or task, and a multi-channel EEG response is recorded. Unique biomarkers are extracted from the recorded EEG response (e.g., as periodogram data points corresponding to different frequencies observed in the brain waves, which can be used to identify a person). A novel signal processing approach uses neural network-based architecture to analyze the EEG response and identify the subject. This signal processing architecture can be readily implemented on hardware and provides high accuracy, precision, and recall.

SVRTEB is universally applicable, as EEG signals can be acquired from all human brains. EEG biometrics cannot be easily forged or collected without consent. Because brainwaves carry unique biomarkers for an individual's response to external stimulus, the proposed model can identify individuals with an accuracy greater the 99%. In addition, SVRTEB can be implemented with commercially available devices, such that the entire process chain can be implemented on a field-programmable gate array (FPGA) or other readily available hardware for real-time identification. Finally, using EEG provides enhanced privacy as EEG signals are difficult to decode and decipher a subject's true identity.

I. Overview of Simple Visual Reaction Task-Based EEG Biometry (SVRTEB)

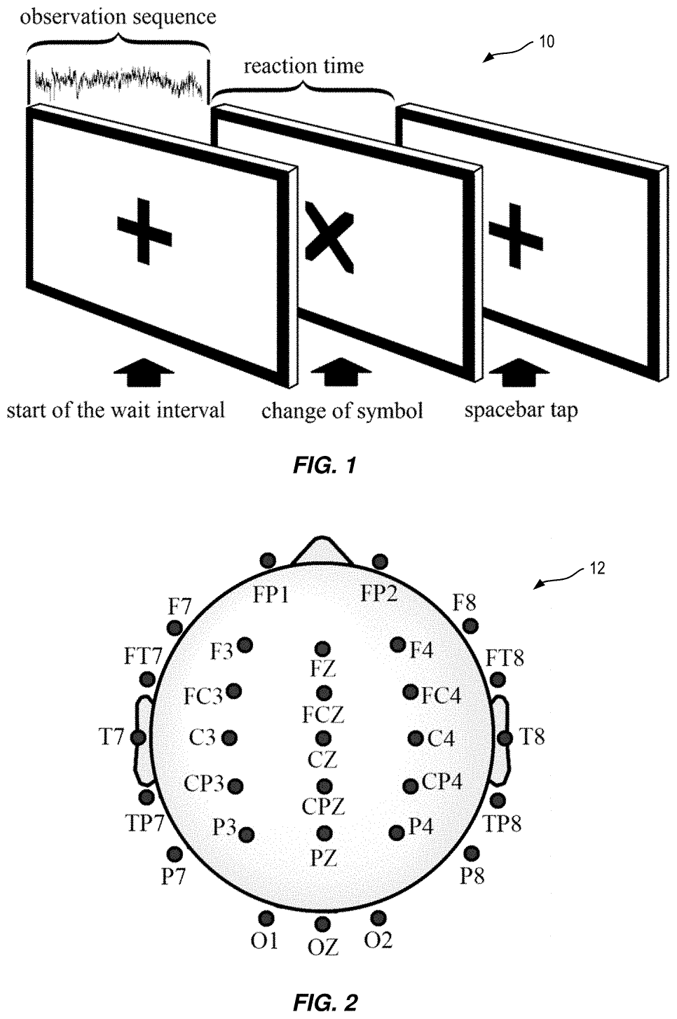

FIG. 1 is a schematic diagram of an exemplary visual response task used to extract unique biomarkers from EEG signals according to embodiments described herein. Some embodiments of SVRTEB are implemented with a computing device (described further below in Section V) which includes a visual output device for presenting visual stimuli to a human subject. An input device, which may be separate from or part of the visual output device, can receive responses from the subject.

For example, the visual output device (e.g., a digital screen) can be placed in front of the subject and present a simple task 10 to be performed. The task 10 may be to respond to a change in the visual output (e.g., changing a sign on the screen from a + symbol to an X symbol) through pressing a button (e.g., a spacebar), tapping a touch screen, or otherwise responding through the input device. In other examples, a stimulus may be provided to the subject or observed by a computer system monitoring the subject.

Embodiments of SVRTEB record multi-channel EEG data during the task 10. This includes recording an observation period before the symbol change and recording a reaction period between the symbol change and performance of the task 10 through the input device. The observation period can render individual attention markers, where an individual's response to external stimulus (e.g., the symbol change) is different and produces unique EEG patterns. Thus, embodiments of SVRTEB extract unique attention markers from the EEG data to identify individuals.

SVRTEB can be used for many applications. The unique attention markers from the EEG can be extracted and analyzed for EEG-based biometry. Reaction-time estimation can be extracted to monitor health and neurological conditions of the subject. In addition, SVRTEB can provide patient monitoring for attention-related disorders.

In further embodiments, the stimulus may be an environmental stimulus (e.g., a change in visual, auditory, or other sensory aspects of an environment observed by the subject and the computer system) or a prompt to interact with a user interface (e.g., to enter a password at the computer system or another device). It should be understood that the tasks/stimuli are not limited to visual tasks or stimuli, but can include a wide variety of tasks or stimuli which result in an observable change in brainwave activity of the subject.

FIG. 2 is a diagram of exemplary electrode locations 12 for recording multi-channel EEG data during the task 10 of FIG. 1. Each electrode in the diagram corresponds to a channel in EEG data received from a subject. In an exemplary aspect, the channels are distinguished using a 2-3 character name. The last character indicates if the channel is in a central line, left hemisphere, or right hemisphere of a subject's brain. The letter Z in the last character indicates the central line, even numbers indicate the right hemisphere, and odd numbers indicate the left hemisphere. The beginning characters of the channel name indicate the channel's position corresponding to different lobes as follows:
F—Frontal Lobe
T—Temporal Lobe
C—Central Lobe
P—Parietal Lobe
O—Occipital Lobe
FC—between F and C
FT—between F and T
CP—between C and P
TP—between T and P

Embodiments of SVRTEB use multi-channel EEG data to enhance analysis of the subject's response to external stimuli and increase biometric identification accuracy. For example, an evaluation of SVRTEB was performed using a non-invasive thirty-channel EEG (described further below in Section III). The EEG data can include multiple trials for a given subject, where a trial includes a stimulus (e.g., visual or otherwise, including multiple types of stimuli) and response.

The visual stimuli provided to a subject can be of different types and forms. For example, the digital screen can provide video sequences with specific objects appearing randomly or complex images with components of interest. In some examples, a natural visual cue can be used, such as any object or person or combination of both. The stimuli may be provided by embodiments of the present disclosure, or it may be externally provided and observed to elicit a response by the subject.

Similarly, responses can be obtained in various forms. The response may be brain activity alone, or it may also include a motor control or other response of the subject. For example, a motor control response can be received through a mouse click, a keyboard press, a buzzer press, a vocal response, or any other action by the subject that can be timed through an input device. In further examples, the motor control response may be observed through sensors (e.g., ocular tracking, motion sensors, and so on).

II. Signal Processing and Analysis Approach of SVRTEB

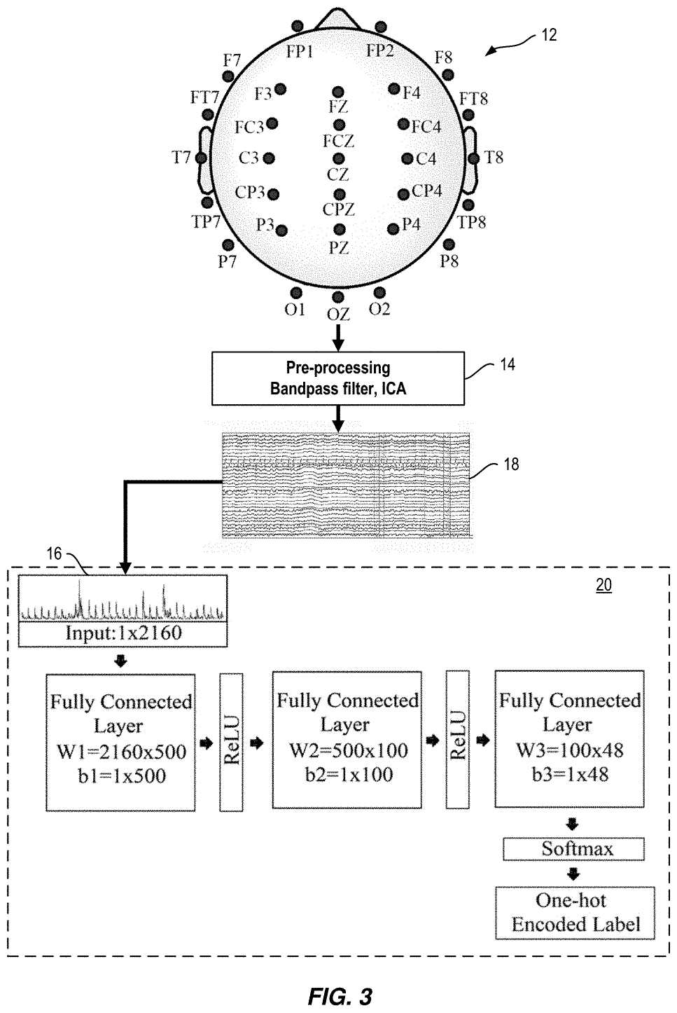

FIG. 3 is a flow diagram of a method for classifying an individual from EEG data according to embodiments described herein. In an exemplary aspect, SVRTEB receives and/or records multi-channel EEG data, such as thirty-channel EEG data produced at the electrode locations 12 of FIG. 2. The multi-channel EEG data is pre-processed 14, which can include applying a bandpass filter as well as applying an independent component analysis (ICA) to remove ocular artifacts (further described in Section II-A below). Next, spectral features 16 are extracted from pre-processed EEG data 18 (further described in Section II-B below). The spectral features 16 are then analyzed using a neural network 20 to classify the individual (further described in Section II-C below).

The method used for SVRTEB illustrated in FIG. 3 provides simple signal processing feature extraction and a neural network architecture which can be implemented real-time on an FPGA.

A. EEG Data Pre-Processing

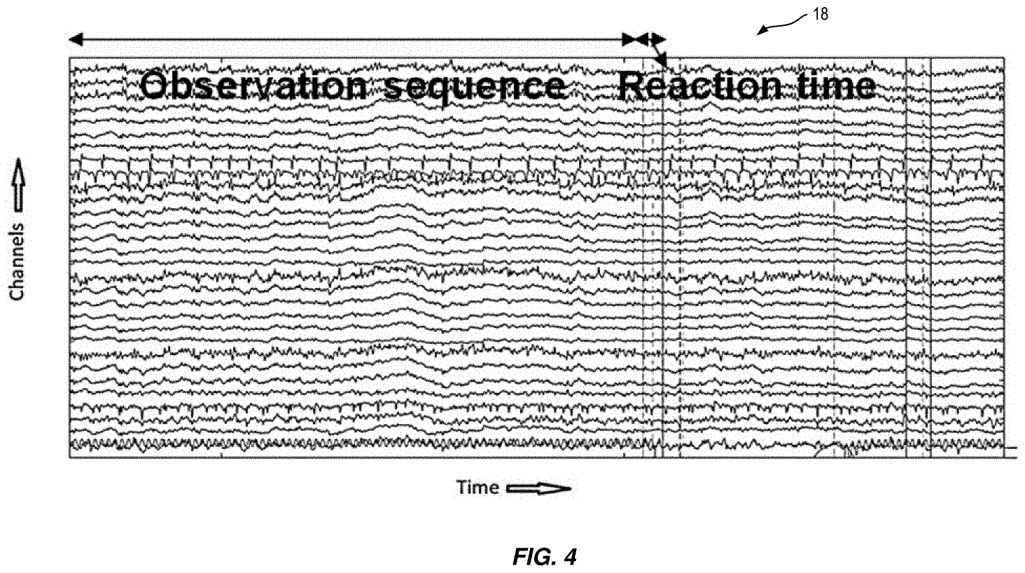

FIG. 4 is a graphical representation of exemplary multi-channel EEG data 18 from the task of FIG. 1 after pre-processing. Raw EEG data 18 (illustrated using the channel names described above with respect to FIG. 2) is digitally converted and marked with stimulus trigger codes (e.g., to indicate the observation period and the reaction period). Pre-processing can include one or more of down-sampling the EEG data 18 (e.g., to 250 hertz (Hz)) and bandpass filtering the EEG data 18 (e.g., between 1 Hz and 35 Hz, which may indicate a range of human brainwaves).

In some embodiments, motor control artifacts (e.g., ocular artifacts such as changes in the EEG data from eye movements) are removed using ICA. In some examples, the down-sampling of the EEG data is performed only for the ICA analysis, with the data being back-projected after motor control (e.g., ocular) artifacts are removed. The EEG data can further be normalized (e.g., by mean and variance) across the channels to remove any DC offset and further analyzed as described below (e.g., using the original sampling rate).

B. Spectral Feature Extraction

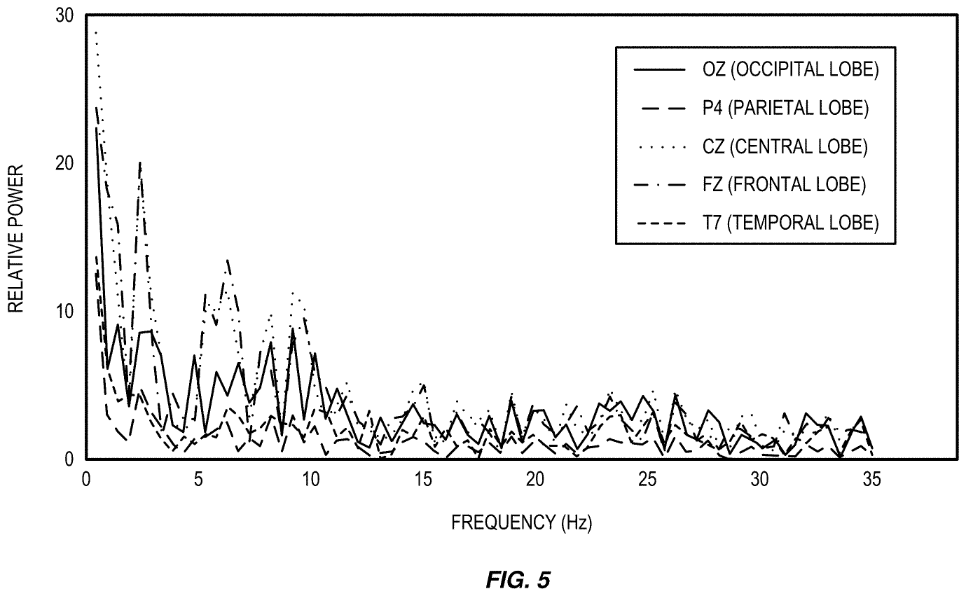

FIG. 5 is a graphical representation of a periodogram from five channels of the multi-channel EEG data 18 of FIG. 3 representing five different brain lobes. For each trial, spectral features can be extracted from the EEG data (e.g., the pre-processed EEG data) during the observation period. These spectral features provide unique biomarkers which identify an individual. For example, periodogram data points can correspond to different frequencies observed in the brain waves that vary from person to person to provide the unique biomarkers.

A number of spectral features can be extracted for each channel of the multi-channel EEG data. For example, the spectral features can include an estimated power spectral density and a square of absolute valued Fourier transform. The feature space contains components from frequency bands between 1 to 35 Hz (e.g., from delta to beta brain wave frequency bands). In some embodiments, the analyzed spectral features include information from delta, theta, alpha, and beta brain wave frequency bands. In some embodiments, information from the gamma brain wave frequency band may also be used.

A number of feature points are extracted for each channel (e.g., having multiple feature points for each spectral feature). In an exemplary embodiment, 72 feature points are obtained from each channel. This results in a total of 2160 feature points (72 feature points×30 channels) from a single trial to be used for classifying a subject. It should be understood that any number of channels may be collected and analyzed, including a single channel.

C. Neural Network Architecture

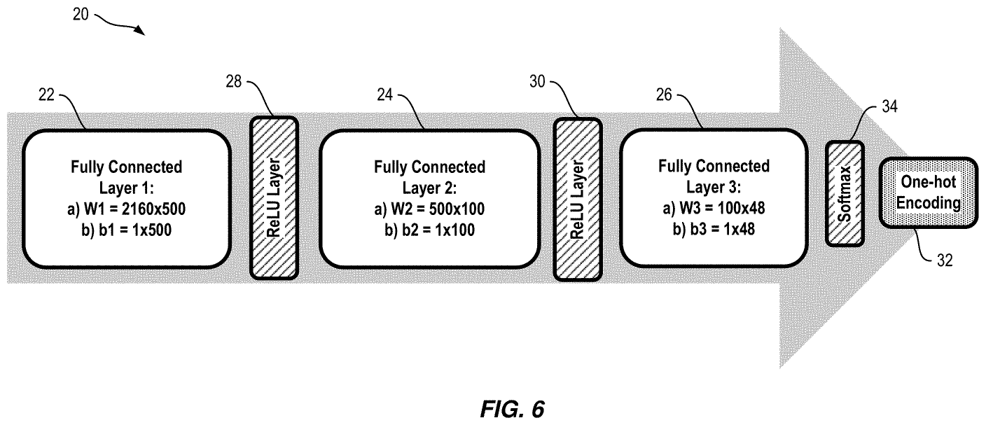

FIG. 6 is a schematic block diagram of an exemplary neural network 20 architecture for classifying an individual from EEG data according to embodiments described herein. In an exemplary aspect, the neural network 20 architecture includes a fully connected neural network (FCNN).

In the example of the FCNN, the neural network 20 includes three fully connected layers 22, 24, 26. A first fully connected layer 22 has dimensions of W1=2160×500 and b1=1×500, a second fully connected layer 24 has dimensions of W2=500×100 and b2=1×100, and a third fully connected layer 26 has dimensions of W3=100×48 and b3=1×48. There are two rectified linear unit (ReLU) layers 28, 30 in between the three fully connected layers 22, 24, 26.

The output of the FCNN is one-hot encoded labels 32. A softmax layer 34 is placed between the third fully connected layer 26 and the one-hot encoded labels 32 to produce a probability distribution of each class. The FCNN uses cross entropy as the loss function of the model.

III. Evaluation

Returning to FIG. 2, the embodiment of SVRTEB used for evaluation used silver/silver chloride (Ag/AgCl) electrodes attached to an elastic cap. The elastic cap brought the electrodes into contact with the head of each human subject. The EEG data was amplified and recorded for each subject, then analyzed using methods described further below. It should be understood that other embodiments of SVRTEB can use more or fewer electrodes, different electrode materials, or otherwise measure and record EEG signals of subjects.

The SVRTEB was evaluated with a total of 15,324 trials from 48 subjects. The entire dataset was split into a training set (65%) and a testing set (35%), with the training and test sets being adequately balanced. Performance of the SVRTEB model was measured based on classification accuracy (%), precision (%), and recall (%). The classification process of SVRTEB described in Section II was repeated for multiple randomizations, where the training and testing sets were randomized anew for each repetition. This measures the sensitivity of the SVRTEB model.

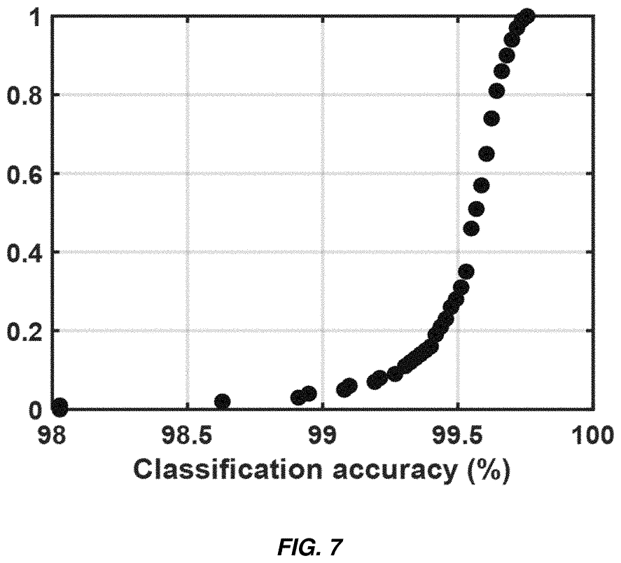

FIG. 7 is a graphical representation of classification accuracy of one hundred randomized Monte Carlo simulations using embodiments described herein. These simulations resulted in an average accuracy of 99.6% (with >99% accuracy in 95/100 simulations), an average precision of 99.7%, and an average recall of 99.3%.

IV. Method for Identifying a Human Subject

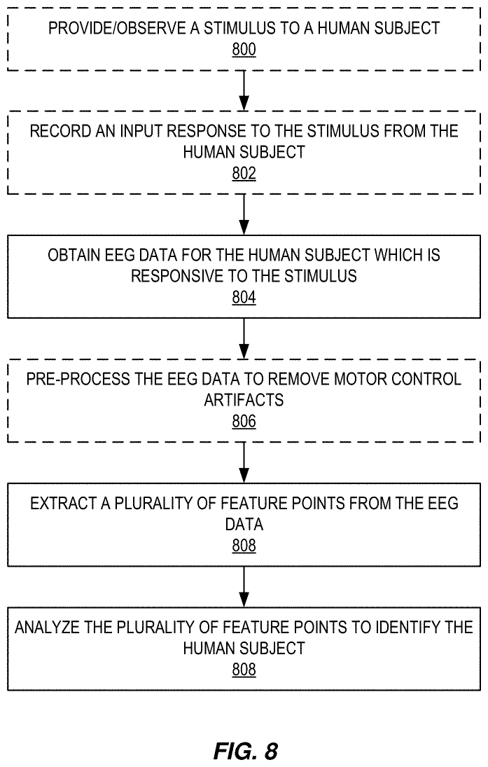

FIG. 8 is a flow diagram illustrating a process for identifying a human subject. Dashed boxes represent optional steps. The process may optionally begin at operation 800, with providing or observing a stimulus (e.g., a visual, auditory, or other stimulus) to a human subject. The process may optionally continue at operation 802, with recording an input response to the stimulus from the human subject. The response may be observed through sensors or a user interface (e.g., an input device). The process continues at operation 804, with obtaining EEG data for the human subject which is responsive to the stimulus.

The process may optionally continue at operation 806, with pre-processing the EEG data to remove motor control (e.g., ocular) artifacts. In an exemplary aspect, the pre-processing may include bandpass filtering the EEG data within a range of human brain waves, down sampling the EEG data (e.g., only for ICA analysis), ICA to remove the ocular artifacts, and/or normalizing the EEG data across each channel of the EEG data. The process continues at operation 808, with extracting a plurality of feature points (e.g., spectral feature points) from the EEG data. The process continues at operation 810, with analyzing the plurality of feature points (e.g., with a neural network) to identify the human subject.

Although the operations of FIG. 8 are illustrated in a series, this is for illustrative purposes and the operations are not necessarily order dependent. Some operations may be performed in a different order than that presented. Further, processes within the scope of this disclosure may include fewer or more steps than those illustrated in FIG. 8.

V. Computer System

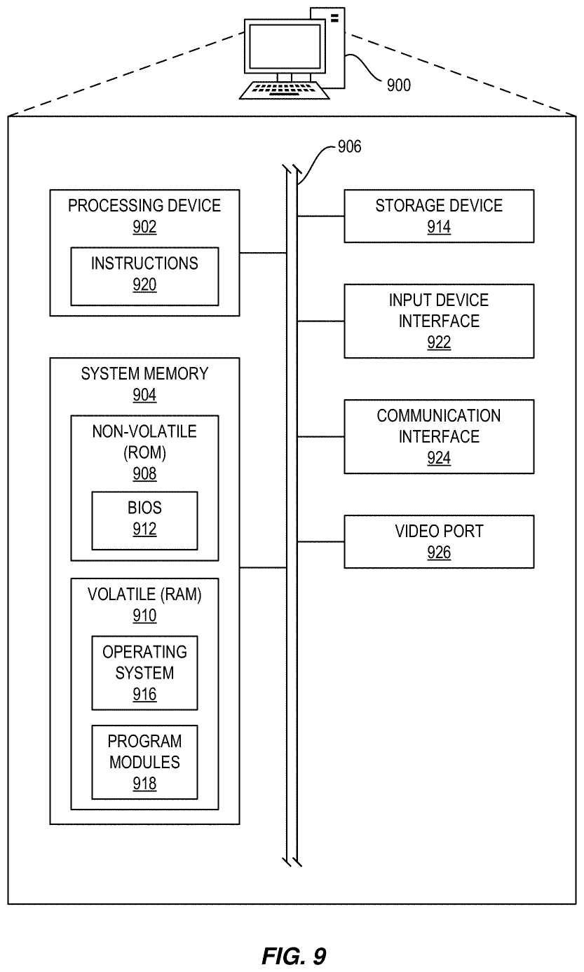

FIG. 9 is a block diagram of a biometric classification device suitable for implementing SVRTEB according to embodiments disclosed herein. The biometric classification device includes or is implemented as a computer system 900, which comprises any computing or electronic device capable of including firmware, hardware, and/or executing software instructions that could be used to perform any of the methods or functions described above, such as identifying a human subject. In this regard, the computer system 900 may be a circuit or circuits included in an electronic board card, such as a printed circuit board (PCB), a server, a personal computer, a desktop computer, a laptop computer, an array of computers, a personal digital assistant (PDA), a computing pad, a mobile device, or any other device, and may represent, for example, a server or a user's computer.

The exemplary computer system 900 in this embodiment includes a processing device 902 or processor, a system memory 904, and a system bus 906. The system memory 904 may include non-volatile memory 908 and volatile memory 910. The non-volatile memory 908 may include read-only memory (ROM), erasable programmable read-only memory (EPROM), electrically erasable programmable read-only memory (EEPROM), and the like. The volatile memory 910 generally includes random-access memory (RAM) (e.g., dynamic random-access memory (DRAM), such as synchronous DRAM (SDRAM)). A basic input/output system (BIOS) 912 may be stored in the non-volatile memory 908 and can include the basic routines that help to transfer information between elements within the computer system 900.

The system bus 906 provides an interface for system components including, but not limited to, the system memory 904 and the processing device 902. The system bus 906 may be any of several types of bus structures that may further interconnect to a memory bus (with or without a memory controller), a peripheral bus, and/or a local bus using any of a variety of commercially available bus architectures.

The processing device 902 represents one or more commercially available or proprietary general-purpose processing devices, such as a microprocessor, central processing unit (CPU), or the like. More particularly, the processing device 902 may be a complex instruction set computing (CISC) microprocessor, a reduced instruction set computing (RISC) microprocessor, a very long instruction word (VLIW) microprocessor, a processor implementing other instruction sets, or other processors implementing a combination of instruction sets. The processing device 902 is configured to execute processing logic instructions for performing the operations and steps discussed herein.

In this regard, the various illustrative logical blocks, modules, and circuits described in connection with the embodiments disclosed herein may be implemented or performed with the processing device 902, which may be a microprocessor, FPGA, a digital signal processor (DSP), an application-specific integrated circuit (ASIC), or other programmable logic device, a discrete gate or transistor logic, discrete hardware components, or any combination thereof designed to perform the functions described herein. Furthermore, the processing device 902 may be a microprocessor, or may be any conventional processor, controller, microcontroller, or state machine. The processing device 902 may also be implemented as a combination of computing devices (e.g., a combination of a DSP and a microprocessor, a plurality of microprocessors, one or more microprocessors in conjunction with a DSP core, or any other such configuration).

The computer system 900 may further include or be coupled to a non-transitory computer-readable storage medium, such as a storage device 914, which may represent an internal or external hard disk drive (HDD), flash memory, or the like. The storage device 914 and other drives associated with computer-readable media and computer-usable media may provide non-volatile storage of data, data structures, computer-executable instructions, and the like. Although the description of computer-readable media above refers to an HDD, it should be appreciated that other types of media that are readable by a computer, such as optical disks, magnetic cassettes, flash memory cards, cartridges, and the like, may also be used in the operating environment, and, further, that any such media may contain computer-executable instructions for performing novel methods of the disclosed embodiments.

An operating system 916 and any number of program modules 918 or other applications can be stored in the volatile memory 910, wherein the program modules 918 represent a wide array of computer-executable instructions corresponding to programs, applications, functions, and the like that may implement the functionality described herein in whole or in part, such as through instructions 920 on the processing device 902. The program modules 918 may also reside on the storage mechanism provided by the storage device 914. As such, all or a portion of the functionality described herein may be implemented as a computer program product stored on a transitory or non-transitory computer-usable or computer-readable storage medium, such as the storage device 914, volatile memory 910, non-volatile memory 908, instructions 920, and the like. The computer program product includes complex programming instructions, such as complex computer-readable program code, to cause the processing device 902 to carry out the steps necessary to implement the functions described herein.

An operator, such as the user, may also be able to enter one or more configuration commands to the computer system 900 through a keyboard, a pointing device such as a mouse, or a touch-sensitive surface, such as the display device, via an input device interface 922 or remotely through a web interface, terminal program, or the like via a communication interface 924. The communication interface 924 may be wired or wireless and facilitate communications with any number of devices via a communications network in a direct or indirect fashion. An output device, such as a display device, can be coupled to the system bus 906 and driven by a video port 926. Additional inputs and outputs to the computer system 900 may be provided through the system bus 906 as appropriate to implement embodiments described herein.

The operational steps described in any of the exemplary embodiments herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary embodiments may be combined.

Those skilled in the art will recognize improvements and modifications to the preferred embodiments of the present disclosure. All such improvements and modifications are considered within the scope of the concepts disclosed herein and the claims that follow.

What is claimed is:

1. A method for identifying a human subject, the method comprising:
obtaining electroencephalogram (EEG) data for a human subject which is responsive to a stimulus;
extracting a plurality of feature points from the EEG data; and
analyzing the plurality of feature points to identify the human subject.

2. The method of claim 1, wherein the stimulus is one or more of a visual stimulus, an audio stimulus, or a sensory stimulus.

3. The method of claim 1, wherein the EEG data comprises motor control data of the human subject responsive to the stimulus.

4. The method of claim 3, further comprising providing the stimulus as a prompt to interact with a user interface.

5. The method of claim 1, wherein extracting the plurality of feature points from the EEG data comprises extracting a plurality of spectral feature points.

6. The method of claim 5, further comprising pre-processing the EEG data to remove motor control artifacts prior to extracting the plurality of spectral feature points.

7. The method of claim 6, wherein the stimulus comprises a visual stimulus and pre-processing the EEG data comprises pre-processing the EEG data to remove ocular artifacts prior to extracting the plurality of spectral feature points.

8. The method of claim 7, wherein pre-processing the EEG data further comprises using independent component analysis (ICA) to remove the motor control artifacts.

9. The method of claim 8, wherein pre-processing the EEG data further comprises:
bandpass filtering the EEG data within a range of human brain waves; and
normalizing the EEG data across each channel of the EEG data.

10. The method of claim 8, wherein pre-processing the EEG data further comprises down sampling the EEG data for the ICA.

11. The method of claim 1, wherein analyzing the plurality of feature points comprises analyzing the plurality of feature points with a neural network to identify the human subject.

12. The method of claim 1, wherein the EEG data comprises multi-channel EEG data.

13. A biometric classification device, comprising:
an electroencephalogram (EEG) sensor;
a memory configured to store EEG data from the EEG sensor; and
a processor configured to:
receive the EEG data for a human subject which is responsive to a stimulus;
extract a plurality of feature points from the EEG data; and
identify the human subject based on the plurality of feature points.

14. The biometric classification device of claim 13, wherein the EEG sensor is a multi-channel EEG sensor.

15. The biometric classification device of claim 13, further comprising a visual output device;
wherein the processor is further configured to cause the stimulus to be displayed via the visual output device.

16. The biometric classification device of claim 15, further comprising an input device configured to receive an input response to the stimulus.

17. A biometric classification system, comprising:
a memory configured to store electroencephalogram (EEG) data from an EEG sensor; and
a processor configured to:
receive the EEG data for a human subject which is responsive to a stimulus;
extract a plurality of feature points from the EEG data; and
implement a neural network to identify the human subject from the plurality of feature points.

18. The biometric classification system of claim 17, wherein the neural network comprises a plurality of fully connected layers and a rectified linear unit (ReLU) layer between the plurality of fully connected layers.

19. The biometric classification system of claim 18, wherein the neural network comprises three fully connected layers and two ReLU layers.

20. The biometric classification system of claim 18, wherein the neural network further comprises one-hot encoding at an output.
